(19) United States
(12) Patent Application Publication
(10) Pub. No.: US 2023/0307126 A1
(43) Pub. Date: Sep. 28, 2023

(54) COMPUTATION OF A QUALITY OF LIFE METRIC

(71) Applicant: INTERNATIONAL BUSINESS MACHINES CORPORATION, Armonk, NY (US)

(72) Inventor: Pritish Ranjan Parida, Cortlandt Manor, NY (US)

(21) Appl. No.: 17/656,478

(22) Filed: Mar. 25, 2022

(51) Int. Cl. G16H 50/20; G16H 10/20

(52) U.S. Cl. CPC G16H 50/20; G16H 10/20

(57) ABSTRACT

A method, computer system, and a computer program for improving a quality of life (QOL) of an individual or a group of individuals is provided. The present invention may include receiving a dataset comprising time-series data pertaining to a QOL factor for the individual or group of individuals. The present invention may then include determining a relative importance of the QOL factor for the individual or group of individuals; computing a QOL metric associated with the individual or group of individuals based on the relative importance of the QOL factor; and determining an intervening measure based on the QOL metric. The present invention may further include requesting an application of the intervening measure based on the QOL metric; and modifying the QOL metric based on a plurality of intervening measure data derived from application of the intervening measure.

### BACKGROUND

The present invention relates generally to the field of patient health, and more particularly, to various embodiments for computing a qualify of life metric reflective of patient health.

Effectively assessing the health and quality of life of an individual proves to be a difficult task due to various factors such as sensitivity of devices acquiring relevant health data, ceiling effect of study groups, and most importantly the lack of a universal and consistent scale used to quantify the individual's health and quality of life. Prior to the advancement of technologies such as wearable devices, data pertaining to the health and quality of life of an individual was limited to readings from medical equipment (e.g., pressure cuff, oximeter, electrocardiogram machine, etc.), the individual's responses to objective questionnaires, and the opinion/diagnosis of the applicable medical professional based on the aforementioned. Although current technologies may serve as a means for centralization of each of the aforementioned data sources, they still do not account for real-time analysis of factors that are imperative to assessing the wearer's quality of life such as pain intensity, sleep quality, mobility, mood, and other relevant factors.

### SUMMARY

Additional aspects and/or advantages will be set forth in part in the description which follows and, in part, will be apparent from the description, or may be learned by practice of the invention.

Embodiments of the present invention disclose a method, system, and computer program product for improving a quality of life (QOL) of an individual or a group of individuals. A computer receives a dataset comprising time-series data pertaining to a QOL factor for the individual or group of individuals and determines a relative importance of the QOL factor for the individual or group of individuals. The computer computes a QOL metric associated with the individual or group of individuals based on the relative importance of the QOL factor and determines an intervening measure based on the QOL metric. The computer requests an application of the intervening measure based on the QOL metric; and modifies the QOL metric based on a plurality of intervening measure data derived from application of the intervening measure. With this embodiment, the QOL factor includes at least one of a mood, a sleep quality, an amount of sleep, a level of mobility, a pain frequency, a pain intensity, or a daily activity associated with the individual or group of individuals.

In accordance with the present invention, the computer correlates the QOL metric to at least one QOL value associated with a questionnaire presented to a user and computation of the QOL metric further includes the computer generating training data from the time-series data; generating a machine-learned output based on the training data; and assigning a plurality of weights to the machine-learned output based on the relative importance.

With this embodiment, the intervening measure is configured to improving the QOL of the individual or group of individuals based on the QOL metric exceeding a threshold value established based on the time-series data via the computer.

### BRIEF DESCRIPTION OF THE DRAWINGS

These and other objects, features, and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates a functional block diagram illustrating a computational environment for a quality of life metric according to at least one embodiment;

FIG. 2 illustrates a block diagram of exemplary network resources of the environment of FIG. 1 according to at least one embodiment;

FIGS. 3A-B illustrates an example set of QOL metric results in accordance with at least one embodiment;

FIG. 4 illustrates a flowchart illustrating a process for computing a quality of life metric according to at least one embodiment;

FIG. 5 depicts a block diagram illustrating components of the software application of FIG. 1, in accordance with an embodiment of the invention;

FIG. 6 depicts abstraction model layers, in accordance with an embodiment of the present invention.

### DETAILED DESCRIPTION

The descriptions of the various embodiments of the present invention will be presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The terms and words used in the following description and claims are not limited to the bibliographical meanings, but, are merely used to enable a clear and consistent understanding of the invention. Accordingly, it should be apparent to those skilled in the art that the following description of exemplary embodiments of the present invention is provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces unless the context clearly dictates otherwise.

It should be understood that the Figures are merely schematic and are not drawn to scale. It should also be understood that the same reference numerals are used throughout the Figures to indicate the same or similar parts.

In the context of the present application, where embodiments of the present invention constitute a method, it should be understood that such a method is a process for execution by a computer, i.e. is a computer-implementable method. The various steps of the method therefore reflect various parts of a computer program, e.g. various parts of one or more algorithms.

Also, in the context of the present application, a system may be a single device or a collection of distributed devices that are adapted to execute one or more embodiments of the methods of the present invention. For instance, a system may be a personal computer (PC), a server or a collection of PCs and/or servers connected via a network such as a local area network, the Internet and so on to cooperatively execute at least one embodiment of the methods of the present invention.

The following described exemplary embodiments provide a method, computer system, and computer program product for improving a QOL of an individual or a group of individuals. Clinical trials, observational studies, and other applicable health-related surveys serve as measurements of health status of a user/patient; however, these are objective and fail to not only effectively integrate real-time collected user specific data necessary to gauge a quantified QOL for the user, but also the aforementioned measurements of health status fail to provide a scalable mechanism configured to serve as a benchmark as to whether the QOL of the user is improving or declining. For example, if a quantified QOL analysis was generated based on the aforementioned trials, studies, and surveys alone then it would not be configured to account for the pain intensity that a patient is experiencing on a frequent and/or daily basis. Lack of accounting for this factor would significantly impact the accuracy of the quantified QOL, especially in instances in which a user has experienced a certain kind of pain for so long that it has become normalized to them. Receiving and processing of both subjective data and objective data in a linear and consistent manner not only allows quantification of an unambiguous QOL metric, but also enables the system to detect the level of impact of QOL factors (e.g., loss of a bodily function, mobility, etc.) and provide remedies and/or solutions to alleviate said factors (e.g., cognitive behavior improvement therapy); thus, increasing the overall QOL of the user. Moreover, exemplary embodiments provided herein allows detection and determination of the relative importance of signals specific to the user necessary to calculate the most reflective QOL metric. In various embodiments, the calculation of the QOL metric and/or components necessary to ascertain the QOL metric are generated by predictive models. Traditionally, predictive models generated by manual techniques (e.g., ad hoc or heuristics) are subject to error; particularly, when the model involves time-series predictive modeling due to the sensitive timeframe associated with the collected data for training data purpose. However, the method, computer system, and computer program product described herein provides mechanisms, such as relevance classification, linear combinations, and correlation to existing medically accepted clinical metrics, and other mechanisms configured to ascertain QOL metrics and provide QOL enhancing intervening measures (e.g., therapy recommendations) in a manner that improves the functioning of a computer by automatically adapting predictive models to solve QOL issues ascertainable from the time-series data. In addition, the invention described herein provides mechanisms such as edge device computation to reduce the amount of data processed and transmitted over a network; thus, reducing the amount of computing resources required to compute large quantities of data needed to ascertain QOL metrics in a scalable manner.

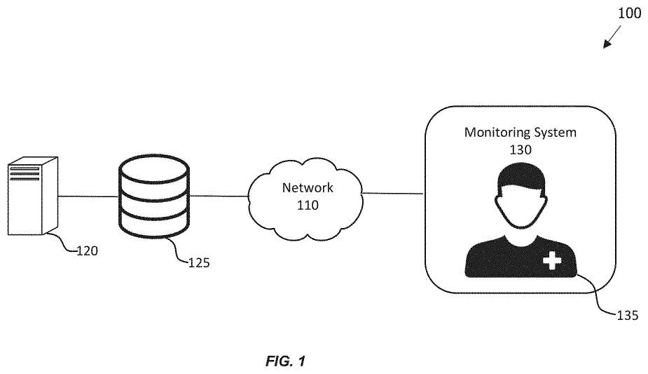

Referring to FIG. 1, a computing system environment 100 for computation and utilization of a QOL metric in accordance with an exemplary embodiment is depicted. FIG. 1 provides only an illustration of implementation and does not imply any limitations regarding the environments in which different embodiments may be implemented. Modifications to environment 100 may be made by those skilled in the art without departing from the scope of the invention as recited by the claims. In this exemplary embodiment, environment 100 includes a server 120 associated with one or more databases 125, and a monitoring system 130 associated with a user/patient and/or group of users/patients 135, each of which are communicatively coupled over a network 110. It should be noted that monitoring system 130 serves the purpose of not only continuously collecting various data associated with user 135, but also identifying a relevance of data acquired from user 135 for the purpose of filtering data transmitted to server 120 by monitoring system 130 over network 110. In some embodiments, monitoring system 130 is a collection of one or more computing devices including or configured to connect to one or more sensors configured to communicate various types of data pertaining to user 135 in order to facilitate monitoring and maintenance of the health of user 135, wherein the various types of data are derived from one or more signals collected by the one or more sensors. The one or more computing devices may include a wearable device, a mobile device, a telephone, a personal digital assistant, a netbook, a laptop computer, a tablet computer, a desktop computer, or any applicable type of computing devices capable of running a program, accessing a network, and/or accessing one or more databases.

As described herein, the various types of data derived from the plurality of signals collected by the one or more sensors and/or applicable questionnaires includes but is not limited to biological data, sleep quality data, pain frequency/intensity data, GPS location, acceleration forces, ambient light/sound, pressure data, body fluid toxicity data, or any other applicable type of data configured to be collected via a computing device and/or sensor known to those of ordinary skill in the art. As described herein, biological data includes but is not limited to heartrate, EEG signals, mood of user 135, blood pressure, eye movement rate, haptic data, body motion data (lack of mobility data), activity data, speech rate, glucose level, respiratory rate, pulse oximetry, breath measurements, skin conductance, or any other applicable data configured to be ascertained from the body known to those of ordinary skill in the art. The one or more sensors may include weight scales, gas detectors, humidity sensors, accelerometers, gyroscopes, barometers, GPS sensors, air quality sensors, ambient sensors, thermometers, haptic sensors, biological-based sensors, or any other applicable sensors known to those of ordinary skill in the art. In some embodiments, computing by one or more edge devices such as the aforementioned computing device allow the plurality of signals to be processed locally (e.g., at the edge device) allowing reduced amounts of data to be received by server 120 and/or the applicable cloud computing environment; thus, not only reducing the amount of computing resources needed to transmit data over network 110, but also increasing the scalability of the centralized platform when handling large quantities of data. For example, the one or more sensors may be a wearable device collecting an accelerometer signal received by the wearable device at 50 Hz from which QOL factors such as effective mobility features can be computed at the edge to produce 1 set of features per hoCur or 1 per day and transferred to the server 120 and/or the cloud.

In some embodiments, server 120 receives a plurality of signals from monitoring system 130 in which the signals are time series signals received by server 120 over a plurality of windows of time. A time series may, for example, be a sequence of data points, measured typically at successive time instants spaced at uniform time intervals. The time series may comprise pairs or tuples reflecting time and value. The values of time series may be derived from sensor data and/or signals. In some embodiments, the signals of a time series may comprise values and a measurand in which the measurand may be a physical quantity, quality, condition, or property being measured. The time series data and/or components of the received plurality of signals are configured to be stored in data records housed on databases 125. It should be noted that the one or more sensors collect the plurality of time series signals in which each time series signal comprises a sequence of values that are captured over time, wherein the source of the plurality of signals varies from implementation to implementation. For example, a first plurality of signals may be derived from a bed sensor of monitoring system 130, while a second plurality of signals may be derived from an accelerator/gyroscope; however, both the first and second pluralities of signals may be within the same window of time in order to ascertain the amount of sleep and the quality of sleep of user 135. In some embodiments, the time series signals may be pre-processed by monitoring system 115 and the time series signals correspond to the plurality of windows of time. It should be understood that the purpose of the plurality of signals being pre-processed is to filter out noise resulting in clean signals being received by server 120. In some embodiments, the time series data may be assigned one or more timestamped scores derived from clinically validated assessments rendered by applicable medical professionals and/or subject matter experts. In some embodiments, one or more components of the timestamped scores may be allocated by server 120 and/or the applicable medical professional or subject matter expert via the centralized platform.

In some embodiments, the plurality of time series signals include a plurality of corresponding labels derived based on one or more extracted features of the signals, in which the corresponding labels are configured to be utilized for model training purposes. However, if a label is not ascertainable for a signal then machine learning clustering methods may be applied to the time series signals. In some embodiments, the plurality of signals includes parameters associated with the biological data/functions monitored by the one or more sensors, wherein the parameters are configured to be utilized for training purposes for one or more models.

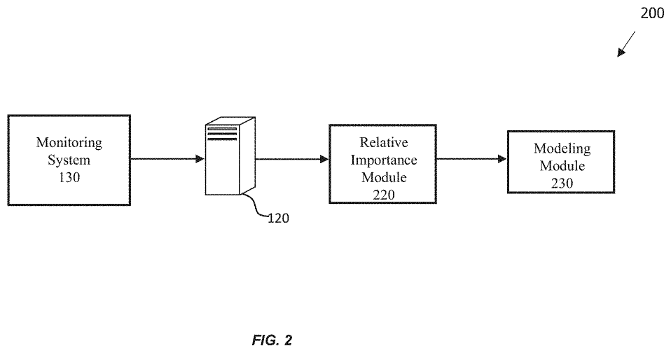

Referring now to FIG. 2, a schematic illustration of exemplary network resources 200 associated with practicing the disclosed inventions is depicted. The inventions may be practiced in the processors of any of the disclosed elements which process an instruction stream. In some embodiments, resources 200 includes a relative importance module 220 and a modeling module 230, wherein modules 220 and 230 are configured to be communicatively coupled to server 120. It should be noted that modules 220 and 230 are configured to utilize machine learning models trained based on data derived from the plurality of signals. In a preferred embodiment, modeling module 230 is configured to train a time series deep learning model; however, selection and filtration of data via server 120 is significantly assisted by the relative importance of data derived from the plurality of signals ascertained by relative importance module 220. It should be noted that relative importance of data ascertained by relative importance module 220 is applied to the data received by server 120 from monitoring system 130 in real time. Relative importance module 220 is configured to utilize gathered data in order to identify one or more QOL factors significantly impacting the QOL of user 135. QOL factors may include but are not limited to pain frequency, pain intensity, sleep quality, mood, daily activities, level of mobility (lack thereof), or any other applicable factor configured to impact one's quality of life. In some embodiments, modeling module 230 may utilize time series deep model learning in order to learn performance of activities of user 135 based on tracking of user 135 via monitoring system 130. The aforementioned learning may be applied based upon modeling module 230 ascertaining the schedule of user 135 via analysis of the day, time, frequency, etc. that user 135 performs activities. Relative importance module 220 ascertains the importance of those activities and the impact of said activities on the quality of life of user 135. For example, based on the plurality of signals received, modeling module 230 may determine that on most mornings user 135 goes for a bicycle ride for a certain window of time, which allows relative importance module 220 to determine that the bicycle rides are of importance to the QOL of user 135. However, the plurality of signals received by the one or more sensors (e.g., accelerometers) and/or responses by user 135 to applicable questionnaires may indicate that the mobility of user 135 during the bicycle rides has been reduced indicating user 135 may be in pain and/or discomfort ultimately indicating a reduction in the quality of life of user 135. Based on the aforementioned, server 120 may prompt user 135, via monitoring system 130, to answer one or more inquiries regarding frequency and intensity of the pain/discomfort that is impacting the bicycle rides. In a preferred embodiment, modeling module 230 utilizes a linear regression model in which the equation for the QOL may be QoL=a0+a1*x1+a2*x2+a3*x3+a4*x4 or in general, Qol=α₀+Σᵢ₌₁^{N}(αᵢxᵢ) where, aᵢ are constants that can be population-based or patient-specific and xᵢ are the different Qol factors.

Modeling module 230 may be a time sequence prediction (e.g., Recurrent Neural Network, Long short-term memory, Convolutional Neural Network, Temporal Convolutional Networks, Temporal Convolutional Network, Encoder-Decoder Temporal Convolutional Network, etc.). Modeling module 230 utilizes the data derived from the plurality of signals to train the machine learned models in order to determine patterns, trends, or any other ascertainable components of the data; simultaneously, relative importance module 220 utilizes one or more machine learned models to not only classify features of the data derived from the plurality of signals, but also designate a label of high importance, moderate importance, low importance, or no importance to the feature associated with the data derived from the plurality of signals. For example, the plurality of signals may be sourced from a bed sensor in which the bed sensor is configured to ascertain occupancy of the bed, the amount of time the bed is occupied, the amount of movement of user 135 in the bed, etc. Meanwhile, one or more sensors associated with monitoring system 130 (e.g., cameras) serves as another source for the plurality of signals which allows the time (e.g, average time within the time series) at which user 135 is waking up to be ascertained, wherein based on a significant distinction between the average time of user 135 waking up and an outlier time indicated from the recently acquired signals, relative importance module 220 can conclude that sleep quality and/or duration of sleep is a QOL factor significantly impacting the QOL of user 135 (e.g., high importance) which must be prioritized in the current calculation of the QOL metric associated with user 135. The QOL factor ascertained from the relative importance module 220 is utilized by modeling module 220 to assign weighting to data points. In particular, relative importance module 220 weighs the contribution of each QOL factor ascertained based on the classification of importance. In some embodiments, classification of the QOL factors allows reduction of computing resources within environment 100 due to the QOL factors allowing server 120 to filter through large quantities of data derived from the plurality of signals in order to optimally train datasets of specific utility to user 135 without wasting unnecessary computing resources. As used herein, the term “training” or “retraining” refers to the process by which a model develops and generates or updating an operating model based on training data, respectively.

It should be noted that modeling module 230 is configured to generate an output of the one or more machine learning models which represents the QOL metric associated with user 135. In some embodiments, each QOL metric generated corresponds to the time series in which the plurality of signals pertains to; however, due to the fact that the data derived from the plurality of signals for the applicable time frame may be sourced from multiple sensors of monitoring system 130 the QOL metric may be a compilation of outputs of machine learning models trained based on the data derived from the plurality of signals. Server 120 may correlate the QOL metric to one or more QOL values associated with questionnaires relating to the health of user 135, such as but not limited to a EuroQol five dimension questionnaire. The terms “EuroQol five dimension questionnaire” and “EQ-5D,” as used herein, refer to a questionnaire that is used to assess the health state (e.g., mobility, self-care, ability to perform usual activities of school, work, or housework, ability to perform ADLs (e.g., dressing, toileting, and cooking), experience of pain or discomfort, and anxiety or depression) of users/patients. Correlation of the QOL metric to one or more values of questionnaires like EQ-5D allow the QOL metric to account for both subjective and objective components relating to mental health, physical health, pain/discomfort levels, daily activities, and mobility/lack thereof associated with user 135. Given the time series of data, modeling module 230 may be used for understanding and/or predicting future data values in the respective time series, which may be accounted for in computation of the QOL metric. Modeling module 230 includes one or more mechanisms that involve modeling adjustments/modifications as a linear combination of time series data (signals) occurring contemporaneously and at various times in the past.

It should be noted that the combination of the relative importance of the QOL factors and the QOL metrics allow server 120 to determine an intervening measure configured to improve the quality of life of user 135. For example, a generated QOL metric may indicate a significant drop in quality of life of the user 135 compared to a previously generated QOL metric in which server 120 is configured to generate one or more intervening measures based on the disparity of the QOL metrics. As described herein, an intervening measure may be any applicable remedy and/or solution configured to improve the quality of life of an individual including but not limited to an increase or decrease of exercise, medication, sleep/rest, change in posture, hobbies (self-care/wellness), treatment (e.g., surgery, therapy, etc.), hygiene, or any other applicable corrective measure or countermeasure known to those of ordinary skill in the art. In some embodiments, upon server 120 determining one or more intervening measures and user 135 implementing/integrating the one or more intervening measures into their life, monitoring system 130 is configured to continue to collect pluralities of signals from the one or more sensors in order for server 120 to derive a plurality of intervening measure data from the one or more intervening measures. The one or more intervening measures are configured to be applied to the datasets housed on databases 125 to be integrated into training sets utilized by modules 220 and 230 for future iterations. The utilization of the intervening measures data by server 120 and/or modeling module 230 allows user 135 to see the impact the intervening measure is contributing to their quality of life. The unconventionality associated with this feature seeks to not only allow user 135 to view the QOL metric, but also the amount of impact QOL factors are having on the QOL metric of user 135.

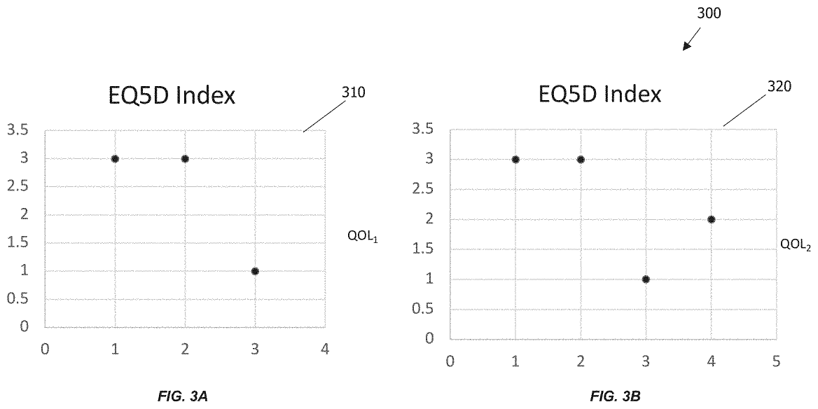

Referring now to FIGS. 3A-B, an example set of QOL metric results 300 in accordance with an exemplary embodiment is depicted. In some embodiments, charts 310-320 depict an example set of time-series data spanning a period of time. For example, chart 310 depicts three consecutive QOL metrics computed which pertain to user 135 in which chart 310 indicates the QOL metric drastically declining. It should be understood that the QOL metric drastically declining may indicate that a QOL factor associated with user 135 is of high importance and said QOL factor is directly impacting the quality of life of user 135. To determine representative QOL metrics, the equations may be the following: QoL0: 5*[(mood-1)/4+(sleepQ-1)/4]; QoL1=5*[(mood-1)/4+(MAX(0,8-ABS(8-Hr))/8)*(sleepQ-1)/4]; QoL2=10*[(mood-1)/4+(MAX(0,8-ABS(8-Hr))/8)*(sleepQ-1)/4+Normalized_eff mob]/3; QoL3=10*[(mood-1)/4+(MAX(0,8-ABS(8-Hr))/8)*(sleepQ-1)/4+Weighted_activity]/3; and Pain States (PStX)=0.5*[Pain Intensity+(10-QoLX)], wherein QoL0 utilizes the mood and sleep quality of user 135, QoL1 utilizes the mood, sleep quality, and number of hours of sleep of user 135, QoL2 utilizes the mood, sleep quality, number of hours of sleep, and effective mobility of user 135, and QoL3 utilizes the mood, sleep quality, number of hours of sleep, and activities of user 135. In some embodiment, pain intensity is not included in the QOL signal due to it not being ascertainable from the plurality of signals; however, PStX may utilize the pain intensity in combination with QOLX. In some embodiments, the range associated with the aforementioned equations is 0 to 10, wherein 0 represents the worst and 10 represents the best allowing the QOL metric to be monotonic value ranging from worst state to best state. It should be understood that the QOL metrics are configured to be correlated against one or more values derived from questionnaires, such as EQ-5D, in order to ascertain the improved QOL metric. In some embodiments, the relationship between pain intensity and effective mobility can be represented as P=α*EM+b, where a and b are constants. For example, a=5 and b=1, in which the values for P go from 0 to 10 and values for effective mobility can go from 0 to 1. The constants a and b could be patient-specific where a=3, b=0.5 for patient A and for patient B, a=−4 and b=9.

As described above, ascertained QOL factors (e.g., user activities, effective mobility, etc.) are configured to be weighted in order to compute an improved QOL metric. In the example depicted via charts 310-320, chart 310 indicates the sharp decline in QOL of user 135 meaning that not only has a QOL factor been determined via relative importance module 220 but also server 120 and/or modeling module 230 may determine the intervening measure necessary to increase the QOL of user 135. Once the intervening measure is applied, server 120 derives the intervening measure data from the intervening measure and transmits the intervening measure data to modeling module 230 for insertion into the one or more machine learned models resulting in the increase in QOL metric reflected in chart 320. It should be understood that there are a multitude of factors as to why the QOL metric may increase; however, progressive iterations performed by modeling module 230 allows for integration of outputs of the machine learned models that include but are not limited to the ascertained QOL factors (including their weighing), previously computed QOL metrics, correlations to objective questionnaires, and any other applicable data configured to be ascertained from the plurality of signals and/or data provided to server 120. Chart 320 depicts a significant increase between QOL1 and QOL2 indicating user 135 more than likely experienced a decrease in pain intensity/frequency and/or increase of mood, quality/quantity of sleep, enhanced mobility, etc. It is imperative to note that stark distinctions between QOL metrics may be based on a plurality of external factors associated with user 135 (e.g., pain/discomfort, unfamiliar sleeping environment, stress levels, etc.); however, the aforementioned equations for computation of data derived from the plurality of signals and/or received by server 120 allowing for consistent health status metric. In some embodiments, mechanisms such as unsupervised learning and rule-based modeling may be used to compute QOL metrics when utilization of modeling module 230 is deemed unnecessary. It is possible for a computed QOL metric to be an average of QOL metrics representative of the time series in which the plurality of signals apply to, wherein acknowledgment of a decrease or increase of the quality of life associated with user 135 is reflected within charts 310-320 as data point outliers. In instances in which the QOL metrics pertain to a group of individuals, the individual data points may represent an average of the group; however, it is it imperative to note these data points do not include the weighing of QOL factors because the data points are objective rather than user-specific.

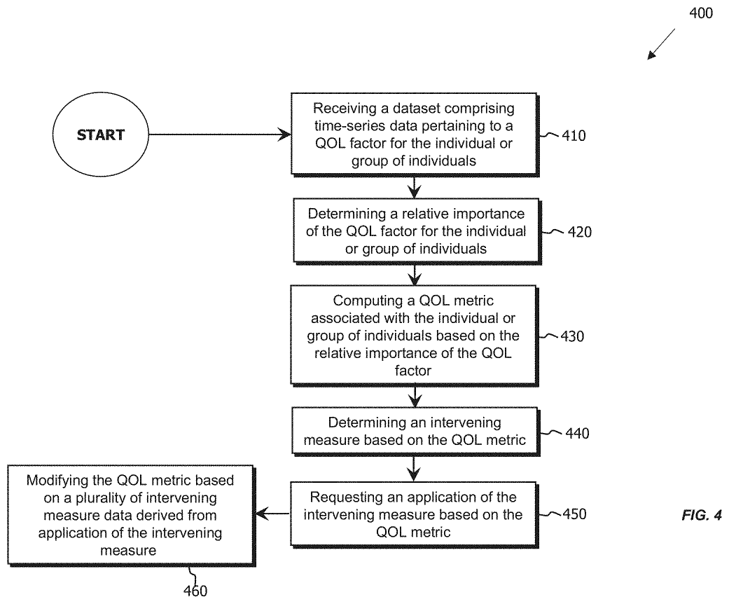

Referring now to FIG. 4, an operational flowchart illustrating an exemplary process for improving a QOL of an individual and/or group of individuals is depicted according to at least one embodiment.

At step 410 of process 400, a dataset comprising time-series data pertaining to a QOL factor for user 135 or group of individuals is received. The plurality of signals received by server 120 from the one or more sensors are collected in the dataset housed within databases 125. In some embodiments, server 120 is configured to generate a centralized platform configured to be utilized by user 135 in which the centralized platform may provide one or more user interfaces to user 135 in order to solicit various types of data not configured to be ascertainable from the one or more sensors. For example, the one or more sensors may not be able to acquire data pertaining to the pain intensity/frequency experienced by user 135; however, the centralized platform may provide to the one or more computing devices of monitoring system 130 user interfaces including prompts, interactive scales, or any other applicable graphical mechanism configured to receive inputs from user 135 regarding their pain intensity/frequency. The data acquired from the centralized platform may be processed by relative importance module 220 in order to determine relevancy and apply classification if necessary, and the resulting data is stored in combination with the data derived from the one or more sensors in databases 125.

At step 420 of process 400, relative importance module 220 determines a relative importance of one or more QOL factors derived from the plurality of signals received by the one or more sensors. In some embodiments, the QOL factors may be detected via server 120 and/or modeling module 230 in which identification and grouping of QOL factors may be accomplished via one or more grouping/clustering algorithms including but not limited to k-means, Euclidean, or any other applicable clustering approach known to those of ordinary skill in the art. In some embodiments, server 120 is configured to apply a classification threshold established based on one or more health-based components within the plurality of signals. For example, the plurality of signals acquired by the one or more sensors may acquire a blood pressure signal associated with user 135 via a first sensor of monitoring system 130 and a lack of sleep signal associated with user 135 via a second sensor of monitoring system 130 both of which may indicate that user 135 is in a stressed state. Modeling module 230 being able to detect QOL factors included within the data derived from the aforementioned signals allows server 120 to transmit the QOL factors to relative importance module 220 for assignment/labeling of relative importance. It should be noted that the relative importance is utilized to assist with correlation of the plurality of signals to the impact of the QOL factors to QOL of user 135 in addition to provide weighing of the data for training purposes. In some embodiments, such as a cloud computing environment, the time-series data of Qol factors can be aggregated and used in conjunction with validated assessments to determine the relative importance of the QOL metric for user 135 or a cohort of patient population. EQ5D-5L, ODI, BDI-II, PCS, FABQ, PPR, PGIC are examples of clinically validated or acceptable assessments. For example, server 120 may compare the scores from clinically validated assessment EQ5D-5L against the QOL factors for a cohort of patient population, and server 120 may determine that overall pain, steps count, effective mobility, mood, and sleep quality are more important than any other Qol factors for user 135. Using the relative importance (which can be captured in the form of relative weights), the time-series data of Qol factors can be combined using a weighted linear combination to generate a time-series of Qol metric. In some embodiments, machine learning algorithms can also be used to identify relative importance of Qol factors.

By applying a time-series forecasting method on the time-series of Qol metric, future values of Qol metric can be predicted for the next hour or next day or next few days or next few weeks. Examples of time-series forecasting method are—Autoregression (AR), Moving Average (MA), Autoregressive Moving Average (ARMA), Autoregressive Integrated Moving Average (ARIMA), Seasonal Autoregressive Integrated Moving-Average (SARIMA), Seasonal Autoregressive Integrated Moving-Average with Exogenous Regressors (SARIMAX), Vector Autoregression (VAR), Vector Autoregression Moving-Average (VARMA), Vector Autoregression Moving-Average with Exogenous Regressors (VARMAX), Simple Exponential Smoothing (SES), Holt Winter's Exponential Smoothing (HWES). Alternately, machine learning algorithms can also be used for Qol metric prediction.

At step 430 of process 400, server 120, with assistance from modeling module 230, computes the QOL metric associated with user 135 and/or the group of individuals based on the relative importance of the QOL factors applied to the data derived from the plurality of signals. In some embodiments, the one or more machine learned models generated by modeling module 230 are configured to integrate one or more of the pain state and/or pain intensity/frequency (or any other applicable data not ascertainable from the plurality of signals) associated with user 135 that he/she is experiencing during the same period of time the time-series data associated with the plurality of signals is accounting for; however, the data pertaining to the pain state and/or pain intensity/frequency may be acquired directly from user 135 via one or more inputs of user 135 received on the centralized platform operating on the computing device. The purpose of the integrating data not ascertainable from the plurality of signals into the machine learned models is to account for QOL factors that may have a significant impact on the QOL of user 135 that are not ascertainable from the one or more sensors of monitoring system 130. QOL factors may include but are not limited to standing, sitting, doing housework (e.g., doing dishes, laundry, cleaning, etc.), exercising, dressing, showing/bathing, eating/drinking/feeding, driving/traveling/commuting, cooking, lying down, going to work, socializing, or any other applicable QOL factors known to those of ordinary skill in the art. Server 120 is configured to determine and/or extract data analytics associated with QOL factors including but not limited to GPS location of user 135 while performing an aforementioned activity, length of time user 135 performs the aforementioned activity, frequency user 135 performs the aforementioned activity, a mood of user 135 during performance of the aforementioned activity. In some embodiments, server 120 is configured to received the data analytics from sources outside of monitoring system 130 including but not limited to web crawlers, 3^{rd} party servers/networks, analytics applications, or any other applicable data sources known those of ordinary skill in the art. For example, a mobile application operating on a computing device may include a feature configured to calculate the level of engagement of user 135 with the mobile application (e.g., eye gaze rate, typing speed, response rate, etc.), in which the level of engagement may be utilized as a QOL factor to be included in the one or more machine learned models. In light of the fact that components of the QOL factors and the data analytics may be ascertainable from the plurality of signals (e.g., heartrate of user 135, location of user 135, etc.), “gaps” in the QOL factors and data analytics may be filled by server 120 via prompting user 135 for inputs on the centralized platform. For example, signals received from the one or more signals of monitoring system 130 may indicate based on the pattern of movements of the user 135 during the period of time that he/she is brushing their teeth; however, server 120 may confirm that this is the activity user 135 is performing via prompting for user 135 to confirm the activity on the wearable presenting the centralized platform. This confirmation allows modeling module 230 to be armed with information such as movements, patterns, and timing associated with when user 135 brushes their teeth for subsequent iterations; hence, when there is a significant disparity in the movements, location, timing, etc. associated with user 135 brushing their teeth, the current computed QOL metric may indicate a decrease in the quality of life in light of one of the previously computed QOL metrics associated with user 135 based on relative importance module 220 labeling user 135 brushing their teeth as a highly important QOL factor. In some embodiments, QOL factors and/or representative values thereof may be ascertained by responses to one or more questionnaires presented to user 135 via the centralized platform.

At step 440 of process 400, server 120, with assistance from modeling module 230, determines an intervening measure based on the QOL metric. The most recently computed QOL metric indicates a disparity from the prior computed QOL metric computed indicating a reduction in the quality of life of user 135 based on the QOL factor. Server 120 is configured to determine an intervening measure configured to curtail the impact caused by the QOL factor. Using the previous example, server 120 may determine that the QOL factor of brushing teeth is impacted due to the range of motion of user 135 being significantly limited (e.g, user 135 sustained injury limiting to his/her range of motion), in which an intervening measure may include user 135 consuming pain medication. In some embodiments, the relationship between the QOL metric and the level of mobility of user 135 is non-monotonic such that the Qol metric deteriorates with level of mobility beyond a certain QOL metric threshold or steps count threshold, then based on the current pain level for the patient a recommendation can be made to either increase or decrease their level of mobility. In some embodiments, the collection of data associated with the aforementioned steps may be presented via the centralized platform to applicable medical professionals and/or subject matter experts for summarization of progress associated with user 135. Medical professionals and/or subject matter experts with qualified access to the centralized platform may provide one or more inputs on computing devices operating the centralized platform in order to facilitate mechanisms such as communication with user 135, supplement data, analyze one or more components and/or sub-components of QOL factors, or any other applicable interactions configured to facilitate the improvement of QOL metrics.

At step 450 of process 400, server 120, with assistance from modeling module 230, requests an application of the intervening measure based on the QOL metric. For example, the QOL metric indicating a reduction in the quality of life associated with user 135 and the determination of server 120 that the intervening measure will assist with curtailing the impact on the quality of life, server 120 requests that application of the intervening measure be applied (e.g., user 135 should consume the pain medication). In some embodiments, server 120 is configured to ascertain the plurality of intervening measuring data from the one or more intervening measures, wherein the plurality of intervening measuring data is continuously being transmitted to the training datasets utilized by the one or more machine learning models. It should be noted that during any of the steps of process 400, server 120 is configured to correlate a previously computed QOL metric and/or a QOL metric currently being generated to QOL values and/or sub-components associated with questionnaires. For example, correlating the computed QOL metric to a value associated with EQ-5D allows a normalization/objectification of the QOL metric in respect to clinical trials, observational studies, etc. In some embodiments, the accuracy of the correlation is confirmed via server 120 based on inputs of the applicable medical professional or subject matter expert provided to the centralized platform.

At step 450 of process 400, server 120, with assistance from modeling module 230, modifies the computed QOL metric based on the plurality of intervening measuring data derived from the application of the intervening measure. It should be understood that the intervening measure data is configured to be received by relative importance module 220 in order to have labels assigned which is necessary for server 120 to determine if the intervening measure is a QOL factor. For example, application of user 135 consuming pain medication may reduce the pain intensity/frequency that user 135 is experiencing when brushing his/her teeth; however, it may not address the lack of mobility of the limbs necessary for user 135 to perform the activity. Thus, it is important for relative importance module 220 to assign the level of importance to the intervening measure in order to assist server 120 in determining if the intervening measure is effective. Modification of the QOL metric may include progression of the QOL metric reflecting the application of intervening measure (e.g. the plurality of intervening measure data indicating an increase in quality of life) and/or re-computation of one or more datapoints into a modified QOL metric allowing the QOL metric to more representative of the window/period of time if applicable.

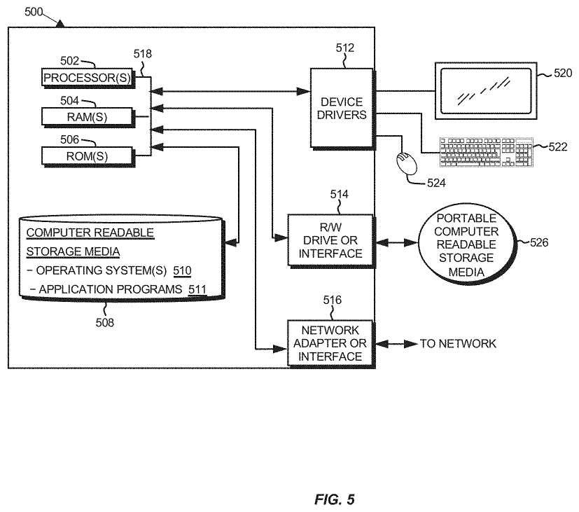

FIG. 5 is a block diagram of components 500 of computers depicted in FIG. 1 in accordance with an illustrative embodiment of the present invention. It should be appreciated that FIG. 5 provides only an illustration of one implementation and does not imply any limitations with regard to the environments in which different embodiments may be implemented. Many modifications to the depicted environments may be made based on design and implementation requirements.

Data processing system 502, 504 is representative of any electronic device capable of executing machine-readable program instructions. Data processing system 502, 504 may be representative of a smart phone, a computer system, PDA, or other electronic devices. Examples of computing systems, environments, and/or configurations that may represented by data processing system 502, 504 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

The one or more servers may include respective sets of components illustrated in FIG. 5. Each of the sets of components include one or more processors 502, one or more computer-readable RAMs 508 and one or more computer-readable ROMs 510 on one or more buses 502, and one or more operating systems 514 and one or more computer-readable tangible storage devices 516. The one or more operating systems 514 and computing event management system 210 may be stored on one or more computer-readable tangible storage devices 516 for execution by one or more processors 502 via one or more RAMs 508 (which typically include cache memory). In the embodiment illustrated in FIG. 5, each of the computer-readable tangible storage devices 516 is a magnetic disk storage device of an internal hard drive. Alternatively, each of the computer-readable tangible storage devices 516 is a semiconductor storage device such as ROM 510, EPROM, flash memory or any other computer-readable tangible storage device that can store a computer program and digital information.

Each set of components 500 also includes a R/W drive or interface 514 to read from and write to one or more portable computer-readable tangible storage devices 508 such as a CD-ROM, DVD, memory stick, magnetic tape, magnetic disk, optical disk or semiconductor storage device. A software program, such as computing event management system 210 can be stored on one or more of the respective portable computer-readable tangible storage devices 508, read via the respective RAY drive or interface 518 and loaded into the respective hard drive.

Each set of components 500 may also include network adapters (or switch port cards) or interfaces 516 such as a TCP/IP adapter cards, wireless wi-fi interface cards, or 3G or 4G wireless interface cards or other wired or wireless communication links. COP 120 can be downloaded from an external computer (e.g., server) via a network (for example, the Internet, a local area network or other, wide area network) and respective network adapters or interfaces 516. From the network adapters (or switch port adaptors) or interfaces 516, computing event management system 210 is loaded into the respective hard drive 508. The network may comprise copper wires, optical fibers, wireless transmission, routers, firewalls, switches, gateway computers and/or edge servers.

Each of components 500 can include a computer display monitor 520, a keyboard 522, and a computer mouse 524. Components 500 can also include touch screens, virtual keyboards, touch pads, pointing devices, and other human interface devices. Each of the sets of components 500 also includes device processors 502 to interface to computer display monitor 520, keyboard 522 and computer mouse 524. The device drivers 512, R/W drive or interface 518 and network adapter or interface 518 comprise hardware and software (stored in storage device 504 and/or ROM 506).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

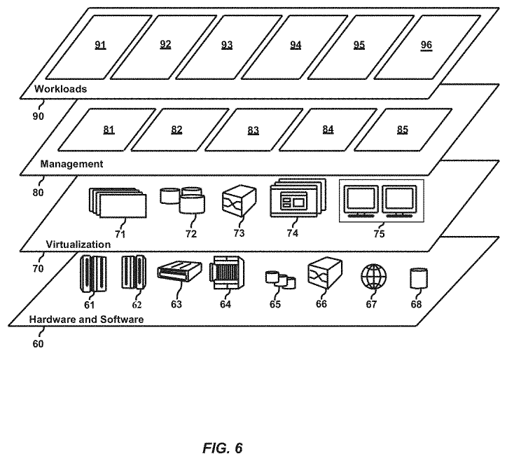

Referring now to FIG. 6 a set of functional abstraction layers provided by environment 100 (FIG. 1) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; and transaction processing 95.

Based on the foregoing, a method, system, and computer program product have been disclosed. However, numerous modifications and substitutions can be made without deviating from the scope of the present invention. Therefore, the present invention has been disclosed by way of example and not limitation.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises,” “comprising,” “includes,” “including,” “has,” “have,” “having,” “with,” and the like, when used in this specification, specify the presence of stated features, integers, steps, operations, elements, and/or components, but does not preclude the presence or addition of one or more other features, integers, steps, operations, elements, components, and/or groups thereof.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

It will be appreciated that, although specific embodiments have been described herein for purposes of illustration, various modifications may be made without departing from the spirit and scope of the embodiments. In particular, transfer learning operations may be carried out by different computing platforms or across multiple devices. Furthermore, the data storage and/or corpus may be localized, remote, or spread across multiple systems. Accordingly, the scope of protection of the embodiments is limited only by the following claims and their equivalent.

What is claimed is:

1. A computer-implemented method for improving a quality of life (QOL) of an individual or a group of individuals, the method comprising:
receiving, via a computing device, a dataset comprising time-series data pertaining to a QOL factor for the individual or group of individuals;
determining, via the computing device, a relative importance of the QOL factor for the individual or group of individuals;
computing, via the computing device, a QOL metric associated with the individual or group of individuals;
determining, via the computing device, an intervening measure based on the QOL metric;
requesting, via the computing device, an application of the intervening measure based on the QOL metric; and
modifying, via the computing device, the QOL metric based on a plurality of intervening measure data derived from application of the intervening measure.

2. The computer-implemented method of claim 1, further comprising:
correlating, via the computing device, the QOL metric to at least one QOL value associated with a questionnaire presented to a user.

3. The computer-implemented method of claim 1, wherein the QOL factor includes at least one of a mood, a sleep quality, an amount of sleep, a level of mobility, a pain frequency, a pain intensity, or a daily activity associated with the individual or group of individuals.

4. The computer-implemented method of claim 1, wherein computing the QOL metric further comprises:
generating, via the computing device, training data from the time-series data;
generating, via the computing device, a machine-learned output from a machine learning model trained with the training data; and
assigning, via the computing device, a plurality of weights to the machine-learned output based on the relative importance of the QOL factor.

5. The computer-implemented method of claim 4, wherein a machine-learned model associated with the machine-learned output is configured to linearly combine the machine-learned output based on the plurality of weights.

6. The computer-implemented method of claim 1, wherein the intervening measure is configured to improve the QOL of the individual or group of individuals based on the QOL metric exceeding a threshold value established, via the computing device, based on the time-series data.

7. The computer-implemented method of claim 1, wherein the QOL metric is configured to be utilized, via the computing device, to determine a level of efficacy of the intervening measure.

8. A computer system for improving a quality of life (QOL) of an individual or a group of individuals, the computer system comprising:
one or more processors, one or more computer-readable memories, and program instructions stored on at least one of the one or more computer-readable memories for execution by at least one of the one or more processors to cause the computer system to:
program instructions to receive a dataset comprising time-series data pertaining to a QOL factor for the individual or group of individuals;
program instructions to determine a relative importance of the QOL factor for the individual or group of individuals;
program instructions to compute a QOL metric associated with the individual or group of individuals;
program instructions to determine an intervening measure based on the QOL metric;
program instructions to request an application of the intervening measure based on the QOL metric; and
program instructions to modify the QOL metric based on a plurality of intervening measure data derived from application of the intervening measure.

9. The computer system of claim 8, further comprising program instructions to:
correlate the QOL metric to at least one QOL value associated with a questionnaire presented to a user.

10. The computer system of claim 8, wherein the QOL factor includes at least one of a mood, a sleep quality, an amount of sleep, a level of mobility, a pain frequency, a pain intensity, or a daily activity associated with the individual or group of individuals.

11. The computer system of claim 8, wherein the program instructions to compute the QOL metric further comprises program instructions to:
generate training data from the time-series data;
generate a machine-learned output based on the training data; and
assign a plurality of weights to the machine-learned output based on the relative importance of the QOL factor.

12. The computer system of claim 11, wherein a machine-learned model associated with the machine-learned output is configured to linearly combine the machine-learned output based on the plurality of weights.

13. The computer system of claim 8, wherein the intervening measure is configured to improve the QOL of the individual or group of individuals based on the QOL metric exceeding a threshold value established, via the computing device, based on the time-series data.

14. The computer system of claim 8, wherein the QOL metric is configured to be utilized to determine a level of efficacy of the intervening measure.

15. A computer program product using a computing device for improving a quality of life (QOL) of an individual or a group of individuals, the computer program product comprising:
one or more non-transitory computer-readable storage media and program instructions stored on the one or more non-transitory computer-readable storage media, the program instructions, when executed by the computing device, cause the computing device to perform a method comprising:
receiving, via the computing device, a dataset comprising time-series data pertaining to a QOL factor for the individual or group of individuals;
determining, via the computing device, a relative importance of the QOL factor for the individual or group of individuals;
computing, via the computing device, a QOL metric associated with the individual or group of individuals;
determining, via the computing device, an intervening measure based on the QOL metric;
requesting, via the computing device, an application of the intervening measure based on the QOL metric; and
modifying, via the computing device, the QOL metric based on a plurality of intervening measure data derived from application of the intervening measure.

16. The computer program product of claim 15, the computing device further configured to:
correlate the QOL metric to at least one QOL value associated with a questionnaire presented to a user.

17. The computer program product of claim 15, wherein computing the QOL metric by the computing device comprises:
generating, via the computing device, training data from the time-series data;
generating, via the computing device, a machine-learned output based on the training data; and
assigning, via the computing device, a plurality of weights to the machine-learned output based on the relative importance of the QOL factor.

18. The computer program product of claim 15, wherein the intervening measure is configured to improve the QOL of the individual or group of individuals based on the QOL metric exceeding a threshold value established, via the computing device, based on the time-series data.

19. The computer program product of claim 15, wherein the intervening measure is configured to improve the QOL of the individual or group of individuals based on the QOL metric exceeding a threshold value established, via the computing device, based on the time-series data.

20. The computer program product of claim 15, wherein the QOL metric is configured to be utilized, via the computing device, to determine a level of efficacy of the intervening measure.
